(19) United States
(12) Patent Application Publication
(10) Pub. No.: US 2007/0238948 A1
(43) Pub. Date: Oct. 11, 2007

(54) System and method to navigate to a slice image in a patient volume data set based on a-priori knowledge and/or prior medical reports

(76) Inventors: Ernst Bartsch, Nurnberg (DE); Thorsten Koopmann, Erlangen (DE); Susanne Laumann, Nuremberg (DE)

Correspondence Address: SCHIFF HARDIN, LLP;PATENT DEPARTMENT, 6600 SEARS TOWER, CHICAGO, IL, 60606-6473, US

(21) Appl. No.: 11/358,200

(22) Filed: Feb. 20, 2006

(51) Int. Cl. A61B 5/05

(52) U.S. Cl. 600/407

(57) ABSTRACT

In a method and system for finding an image slice in a volume data set obtained from a scanner which scans a region of interest of a patient having a medical problem where that medical problem is located within said region of interest, the patient is scanned creating a patient volume data set. At least one of information types prior medical reports of said patient or a-priori knowledge of the type of medical problem are input to a natural language processor. With the natural language processor analyzing the information types to find a most likely volume of interest. The volume of interest is supplied to a navigation component which also receives the volume data set from the scanner, the navigation component also creating landmarks of human anatomy lying within the patient region from the volume data set. The navigation component uses the landmarks, the volume of interest, and the volume data set to identify an image slice of the volume data set which shows a location of the medical of a patient in said region.

### BACKGROUND

The present disclosure relates to medical imaging. When reading follow-up studies, radiologists first need to find a slice image for a patient in volume data sets obtained from major medical imaging modalities (e.g. CT Computer Tomography) and MR (Magnetic Resonance)) of the patient. This has been a manual process previously.

Let it be assumed for example, a previous finding was a lung tumor. In a follow-up study a first step is to find the lung tumor again by scrolling to the right image slice position. Once the previous tumor is found again, the radiologist compares the prior and follow-up study and assesses if e.g. the tumor has grown and if yes, to what extent.

Currently more and more images are produced in less and less time with ever increasing quality by newer modalities. This trend leads to a data/image overload for the radiologist. Automations in reducing reading time are urgently needed.

Currently, radiologists manually scroll to the slice image interest. This is a time consuming task.

### SUMMARY

It is an object to provide an automated solution to find a slice image of interest in medical imaging during follow-up medical studies.

In a method and system for finding an image slice in a volume data set obtained from a scanner which scans a region of interest of a patient having a medical problem where that medical problem is located within said region of interest, the patient is scanned creating a patient volume data set. At least one of information types prior medical reports of said patient or a-priori knowledge of the type of medical problem are input to a natural language processor. With the natural language processor analyzing the information types to find a most likely volume of interest. The volume of interest is supplied to a navigation component which also receives the volume data set from the scanner, the navigation component also creating landmarks of human anatomy lying within the patient region from the volume data set. The navigation component uses the landmarks, the volume of interest, and the volume data set to identify an image slice of the volume data set which shows a location of the medical of a patient in said region.

### DESCRIPTION OF THE DRAWINGS

FIG. 1 shows an image slice of a particular slice number for a patient from a scan of a portion of the human anatomy of the patient and wherein this slice number and corresponding slice image are located automatically with the method and system of the preferred embodiment;

FIG. 2 is a block diagram showing a method and system of the preferred embodiment for automatic navigation to a slice image of interest based on a-priori knowledge and prior medical reports; and

FIG. 3 is a perspective view of the human anatomy showing feature landmark extraction in an axial direction used in the method and system of FIG. 2.

### DESCRIPTION OF THE PREFERRED EMBODIMENT

For the purposes of promoting an understanding of the principles of the invention, reference will now be made to the preferred embodiment illustrated in the drawings and specific language will be used to describe the same. It will nevertheless be understood that no limitation of the scope of the invention is thereby intended, such alterations and further modifications in the illustrated device, and/or method, and such further applications of the principles of the invention as illustrated therein being contemplated as would normally occur now or in the future to one skilled in the art to which the invention relates.

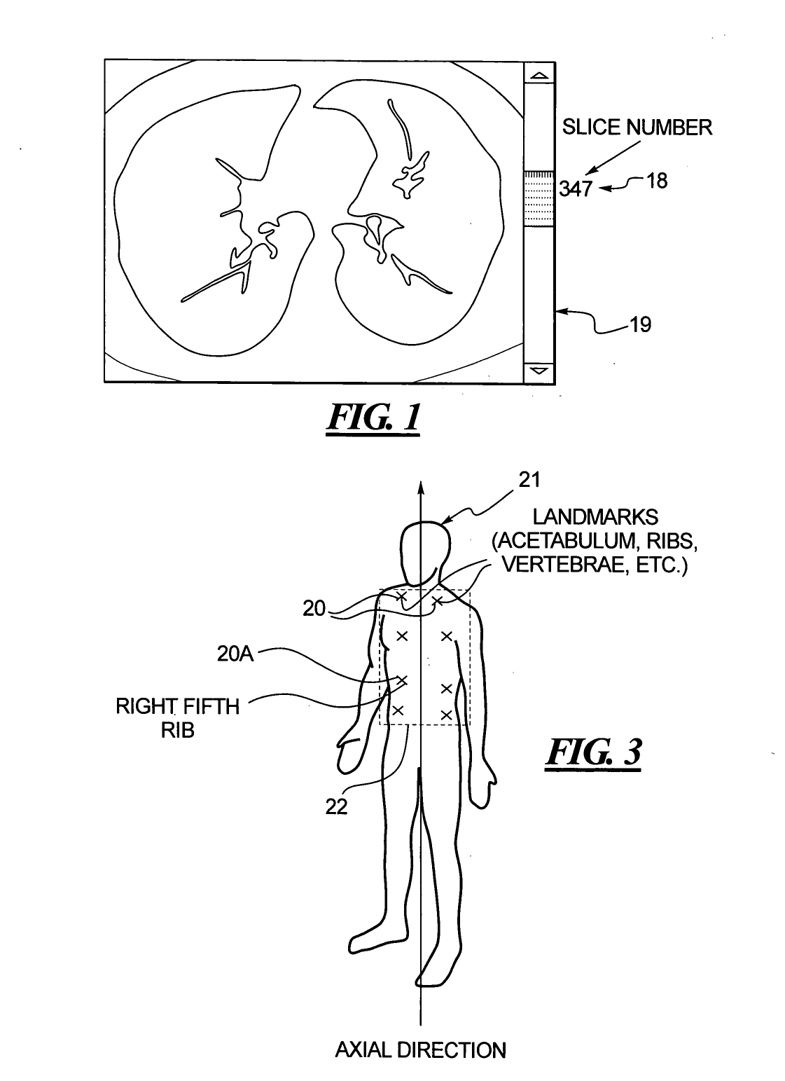

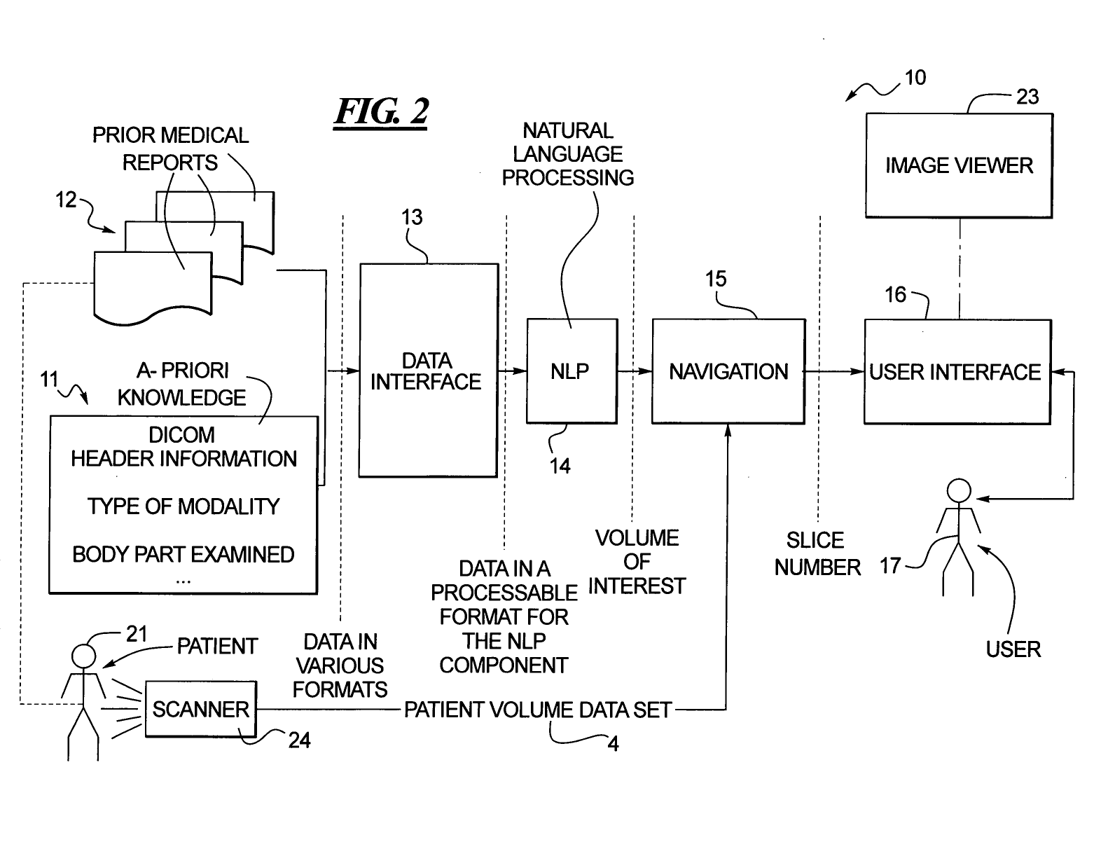

An important concept of the preferred embodiment as shown in FIG. 2 is to automatically navigate the user 17 to a slice image of interest based on a-priori knowledge 11 of the type of medical problem which the patient has encountered, and/or prior specific medical reports 12 of the patient, and a volume data set from a scanner 24 which has scanned region 22 of the patient within which lies a specific location of the patient's specific medical problem. By a-priori knowledge it is meant any previous information or knowledge available on the type of medical problem facing the particular patient. The proposed system 10 and method of FIG. 2 automatically points the user 17 to a correct slice number 18 of a slice image 19 shown in FIG. 1 illustrating the patient's medial problem at a specific location within said region 22 of the patient 21. No manual scrolling is necessary with the preferred embodiment, thus resulting in significant time savings and thereby reducing reading time.

A navigation component 15 finds the right slice position/number based on an anatomic volume of interest supplied by a natural language processing (NLP) component 15.

When opening a study in the image viewer 23, the user 17 is pointed automatically to the right slice number 18 without manual intervention.

During a follow-up study the proposed system automatically navigates the user to the slice image of interest. Only small fine tuning might be necessary; however the user does not have to scroll through hundreds of slices to find the slice image of interest. This automation results in increased work efficiency and the radiologists and/or the cardiologists save considerable time during follow-up studies. A solution for efficient high volume is thus provided.

Technical benefits of the preferred embodiment are that it may be used to load only the slice images of interest to the image viewer 23 and avoid an overload of the network. To explain the preferred embodiment, let it be assumed that the following sample prior medical report of the patient 21 is provided:
“Chest Moderate eventration of the anterior portion of the right hemidiaphragm is present. Partial collapse of the right middle lobe is identified and an unusual spherical density is seen over the apex of the right hemidiaphragm on the PA projection.

A follow-up examination in 10-14 days is highly recommended.”

At the follow-up examination in 10-14 days, the system identifies the right middle lobe and the right hemidiaphragm as the volume of interest in the CT chest scan. Using the disclosed system the chest radiologist is automatically navigated to the volume of interest, i.e. to the right slice number 18 of the slice image 19 of FIG. 1, which shows as an example a CT (computer tomography) chest scan, automatically navigated to the right middle lobe (i.e. slice number 347—slice image 19).

The radiologist can immediately start image reading at the right slice image. This automation saves him/her time.

FIG. 2 illustrates the system of the preferred embodiment for finding a particular slice image for particular patients.

Data sources for the NLP (Natural Language Processing) component 14 are prior medical reports 12 of that patient 21 and a-priori knowledge 11 of the specific medical problem facing that patient 21 and gained from e.g. the DICOM header information, like type of the modality, body part examined, etc. for that patient 21 (DICOM is a standard for medical imaging known as Digital Imaging and Communication in Medicine). This knowledge, together with the prior reports, is processed after data conversion by the NLP module 14 to come up with a most likely volume of interest.

A data interface 13 converts the various data formats into a processable format for the NLP component 14. Additionally it provides a common data interface to various data sources. For example, the data interface 13 could be a parser converting e.g. HTML, windows Microsoft Word format etc. into a format which is readable by the NLP.

The NLP component 14 analyzes the prior medical reports 12 and a-priori knowledge 11 to find a most likely volume of interest. It does this, for example, by analyzing unstructured text information. It looks for key words to identify the medical problem at hand and to identify the anomatical area of interest, e.g. the body part or organ relating to that medical problem.

The navigation component 15 employs feature landmark extraction e.g. in an axial direction (FIG. 2). Other directions, of course, can also be employed. Feature points, contours, and regions are extracted from the volume data set 4 from scanner 24 and used as landmarks 20 on the human anatomy 21 as shown in FIG. 3. This volume data set 4 is provided as an input to the navigation component 15 from a scanner 24 for example an MRI scanner, CT scanner, etc. which scans the region 22 of the patient 21. Thus this volume data set represents the scanned FIG. 1 region 22 at which the medical problem is located. These landmarks 20 should be robust against noises and variations and should be prominent and reliable. There should also be plenty of landmarks 20 that cover the complete volume data set relating to the region 22 shown with dashed lines of the human anatomy of the patient 21 using the previous example of the partial collapse of the middle lobe in the patient's chest. Thus the volume region 22 scanned and shown in FIG. 1 covers the chest and makes up the volume data set. Now it is necessary for the navigation component (e.g. a computer with software) to use the landmarks. In this example, a prominent landmark could be the ribs at landmark 20A.

Using landmarks 20, the navigation component 15 finds the right slice number 18 (FIG. 1), which corresponds to the identified volume of interest supplied by the NLP component 14. Continuing with the previous example, let us assume that the collapse of the right middle lobe in the chest is located very close to the fifth rib on the right side of the patient. One of the landmarks 20A is at this fifth rib. Now the navigation component analyzes the volume data set 4 to find the landmark fifth rib 20A. Now the navigation component looks through for example hundreds of slices of the volume data set 4 and finds the particular slice 347.

The slice number 18 is output to the user 17 with interface 16, which automatically jumps to the given slice number when the radiologist opens the corresponding follow-up study. The image viewer 23 shows the desired image slice 19 called up on user interface 16 with the slice number.

While a preferred embodiment has been illustrated and described in detail in the drawings and foregoing description, the same is to be considered as illustrative and not restrictive in character, it being understood that only the preferred embodiment has been shown and described and that all changes and modifications that come within the spirit of the invention both now or in the future are desired to be protected.

1. A method for finding an image slice in a volume data set obtained from a scanner which scans a region of interest of a patient having a medical problem where that medical problem is located within said region of interest, comprising the steps of:
scanning the patient at said region of interest containing the patient's medical problem and creating a patient volume data set;
inputting at least one of information types prior medical reports of said patient or a-priori knowledge of the type of medical problem which the patient has to a natural language processor;
with the natural language processor analyzing said information types input thereto to find a most likely volume of interest by looking for key aspects of said information types to identify the medical problem and identify an anatomical area of interest relating to that medical problem as said volume of interest; and
supplying said volume of interest to a navigation component which also receives said volume data set from said scanner, said navigation component also creating landmarks of a human anatomy lying within said patient region from said volume data set, said navigation component using said landmarks, said volume of interest from said natural language processing, and said volume data set to identify an image slice of said volume data set which shows a location of the medical problem of the patient in said region.

2. A method of claim 1 wherein said information types comprise both said prior medical reports and said a-priori knowledge.

3. A method of claim 1 wherein said navigation component outputs a slice number of said image slice.

4. A method of claim 1 wherein a user utilizes said slice number to show said slice image on an image viewer.

5. A method of claim 1 wherein said a-priori knowledge comprises DICOM image header information.

6. A method of claim 1 wherein said a-priori knowledge comprises a type of modality.

7. A method of claim 1 wherein said a-priori knowledge comprises body parts examined.

8. A method of claim 1 wherein said at least one information type is input to said natural language processor via a data interface.

9. A method for finding an image slice in a volume data set obtained from a scanner which scans a region of interest of a patient having a medical problem where that medical problem is located within said region of interest, comprising the steps of:
scanning the patient at said region of interest containing the patient's medical problem and creating a patient volume data set;
inputting at least one of information types prior medical reports of said patient or a-priori knowledge of the type of medical problem which the patient has to a natural language processor;
with the natural language processor analyzing said information types input thereto to find a most likely volume of interest; and
said navigation component using said volume of interest from said natural language processing and said volume data set to identify an image slice of said volume data set which shows a location of the medical problem of the patient in said region.

10. A system for finding an image slice in a volume data set obtained from a scanner which scans a region of interest of a patient having a medical problem where that medical problem is located within said region of interest, comprising:
said scanner scanning the patient at said region of interest containing the patient's medical problem and creating a patient volume data set;
a natural language processor to which is input at least one of information types prior medical reports of said patient or a-priori knowledge of the type of medical problem which the patient has;
with the natural language processor analyzing said information types input thereto to find a most likely volume of interest; and
said volume of interest being supplied to a navigation component which also receives said volume data set from said scanner, said navigation component also creating landmarks of a human anatomy lying within said patient region from said volume data set, said navigation component using said landmarks, said volume of interest from said natural language processing, and said volume data set to identify an image slice of said volume data set which shows a location of the medical problem of the patient in said region.

11. A system of claim 10 wherein said information types comprise both said prior medical reports and said a-priori knowledge.

12. A system of claim 10 wherein said navigation component outputs a slice number of said image slice.

13. A system of claim 10 wherein said slice number is employed to show said slice image on an image viewer.

14. A system of claim 10 wherein said at least one information type is input to said natural language processor via a data interface.

15. A computer program product for use in a computer for finding an image slice in a volume data set obtained from a scanner which scans a region of interest of a patient having a medical problem where that medical problem is located within said region of interest, said scanner scanning the patient at said region of interest containing the patient's medical problem and creating a patient volume data set, and wherein at least one of information types prior medical reports of said patient or a-priori knowledge of the type of medical problem which the patient is input to a natural language processor, which in turn connects to a navigation component, said computer program product performing the steps of:
analyzing said information types ito find a most likely volume of interest; and
creating landmarks of a human anatomy lying within said patient region from said volume data set, and using said landmarks, said volume of interest, and said volume data set identifying an image slice of said volume data set which shows a location of the medical problem of the patient in said region.

16. A computer program product of claim 15 wherein said information types comprise both prior medical reports and said a-priori knowledge.
